(19) United States
(12) Patent Application Publication
(10) Pub. No.: US 2026/0157863 A1
(43) Pub. Date: Jun. 11, 2026

(54) COVER FOR A PROSTHETIC HAND AND METHOD FOR PRODUCING SAME

(71) Applicant: OTTO BOCK HEALTHCARE PRODUCTS GMBH, Wien (AT)

(72) Inventors: Walter LUNZER, Wien (AT); Sonja WAGNER, Wien (AT); Angelika WILLCOCK, Wien (AT)

(21) Appl. No.: 18/706,584

(22) PCT Filed: Nov. 9, 2022
(86) PCT No.: PCT/EP2022/081314

§ 371 Date: May 1, 2024

(30) Foreign Application Priority Data
Nov. 17, 2021 (DE) 10 2021 130 037.1

(51) Int. Cl. A61F 2/58; A61F 2/50

(52) U.S. Cl. CPC A61F 2/586; A61F 2/5044; A61F 2002/5001

(57) ABSTRACT

The invention relates to a cover for a prosthetic hand, having a textile main part that covers at least a palm area of a prosthetic hand when worn. The main part is designed as a partial glove having through-openings for prosthetic fingers and at least one distal finger cap consisting of a non-textile plastic material replaces a missing textile finger area.

The invention relates to a cover for a prosthetic hand with a textile main body, which cover at least partially covers at least one palm region of a prosthetic hand in the applied state, and to a method for producing a cover for a prosthetic hand with a main body of this type.

Covers for prosthetic hands are used to cover order to protect a prosthetic hand or components of a prosthetic hand. Here, the covers are of flexible and as a rule elastic configuration with the use of different materials. Firstly, the cover for a prosthetic hand fulfills a protective function, by it protecting the prosthetic hand against external influences, for example dust, scratches and liquids, and at the same time providing padding for natural limbs or objects if contact occurs during the use of the prosthesis. Secondly, the cover for a prosthetic hand fulfills an esthetic function. Users of a prosthesis frequently do not wish it to be recognized as such directly. Finally, covers for prosthetic hands can be a functional component of a prosthetic supply and can provide restoring forces in the case of an elastic configuration, which restoring forces assist drives or brake movements and provide suitable contact surfaces during the use of the prosthetic hand.

EP 2 285 314 B1 discloses a cover for a prosthetic hand with an inner layer and an outer layer, the outer layer being formed from silicone, and the inner layer being formed from an elastane material or polyamide, in particular nylon.

DE 10 2016 003 862 A1 relates to a cover for a prosthetic hand consisting of a front part and a rear part which are connected to one another at one or more joints. The cover for a prosthetic hand has silicone appliqués which correspond to the folds of a human hand. Moreover, fingernail components made from a hard plastic are arranged in each case on the fingers.

Covers for prosthetic hands have up to now been manufactured completely from a silicone material or a PVC material and are configured as a complete glove. As an alternative, covers for prosthetic hands are produced from a combination of a textile layer with a layer of silicone, or are formed from elastic textiles with a complete or partial anti-slip coating. It is a disadvantage of this that the covers for prosthetic hands which are manufactured completely from silicone material or PVC material and the covers for prosthetic hands which are produced from a plurality of layers with different materials impair the adjusting speed, the grip pattern and grip strength of the hand prosthesis and are comparatively heavy. Covers of this type for prosthetic hands additionally have the disadvantage that it is not possible to change them without destruction. Covers for prosthetic hands made from elastic textiles with a complete or partial anti-slip coating have the disadvantage, in contrast, that they afford limited protection against external influences.

It is an object of the present invention to provide a cover for a prosthetic hand and a method for producing a cover for a prosthetic hand, by way of which the prostheses are protected as satisfactorily as possible against external influences with improved functionality and which covers can be replaced by the patients themselves.

According to the invention, this object is achieved by way of a cover for a prosthetic hand with the features of the main claim, and by way of a method with the features of the further independent claim. Advantageous refinements and developments of the invention are disclosed in the subclaims, the description and the figure.

The cover for a prosthetic hand with a textile main body, which cover at least partially covers at least one palm region of a prosthetic hand in the applied state, provides that the main body is configured as a part glove with passage openings for prosthetic fingers, and at least one distal finger cap with a non-textile plastic replaces a missing textile finger region. The functionally and visually most exposed part of a hand prosthesis is the fingers, in particular the finger caps, the distal phalanx and, if present, the middle phalanx, since, during the use of the prosthesis, these regions as a rule come into contact first with other objects, substances and/or other influences. The modular configuration of the cover for a prosthetic hand with at least one distal finger cap which is attached and fastened to the free and open end of a main body which is configured in the form of a part glove and is made from a textile has the advantage that the regions which are loaded or affected most by external influences can be protected in a loading-suitable manner. The finger cap or the finger caps can be manufactured in an individually adapted manner and can be attached to the main body. Here, the functionality of each individual finger cap can be set or selected separately. For instance, a different wall thickness, material characteristic or a different construction can be selected for the finger cap of the index finger than for the thumb or the little finger. At the same time, in addition to the function, the mobility of the prosthesis is not restricted, since there is a flexible and possibly elastic textile main body in the remaining regions of the cover for a prosthetic hand. As a result of the textile main body, the cover for a prosthetic hand can be put on and taken off by the patients themselves and, moreover, is washable. Covers for prosthetic hands which comprise exclusively an elastomer have not been able up to now to be put on and taken off by the patients without outside help. Moreover, a re-use of a cover for a prosthetic hand which is made exclusively from an elastomer and has been removed once is not possible, since such covers are destroyed while being taken off. Covers of this type for prosthetic hands as a rule have to be cut open, in order to remove them from a prosthetic hand. By way of the covers according to the invention for prosthetic hands, everyday handling is substantially facilitated, and they can be readily put on and taken off without destruction by the user themselves.

One development provides that distal finger caps are arranged on all fingers. This refinement has the advantage that the distal ends of all fingers are protected by way of the finger caps and, in addition, a uniform external appearance of the cover for a prosthetic hand is produced.

The respective distal finger cap is preferably sewn, adhesively bonded, cast, printed and/or welded on the textile main body. This affords the advantage that the finger cap does not have to be removed or positioned manually each time that the cover for a prosthetic hand is taken off or put on. In addition, consistent seating of the finger cap on the main body is ensured, with the result that the protection against external influences cannot be lost as a result of the finger cap being positioned incorrectly. Moreover, the finger caps cannot rotate while being worn.

One development provides that the finger cap is removable and/or replaceable. In this embodiment, there is the possibility for individual finger caps to be swapped if they have high wear or visual defects. Therefore, a replacement of the entire cover for a prosthetic hand is not required. If the finger caps are of removable design, simple cleaning of them can additionally be ensured. The fastening of the finger cap to the main body takes place, for example, via hook and loop fastener elements, screws, clamping strips, undercuts or via other positively locking elements.

The distal finger cap is advantageously fastened to a textile sleeve portion of the main body, which sleeve portion extends along the prosthetic finger in the applied state. The sleeve portion is a continuation of the main body and, in the applied state, extends peripherally around the prosthetic finger, but not as far as its distal end. As a result, there is a simple interface structure between the main body and the finger cap, which interface structure can be manufactured and mounted with low complexity. This sleeve portion preferably extends as far as a first or second finger joint of the prosthetic finger in the applied state. In this embodiment, the first and/or the second finger joint of the prosthetic hand can move in an unrestricted manner.

One development provides that at least one reinforcing element is stitched, sewn, adhesively bonded, cast, printed and/or welded on the main body. A reinforcing element or patch of this type can be produced, for example, from textile, leather or PU foam, and can improve the contact of the finger cap with the prosthetic finger, in particular when a flexion or extension of a joint takes place at this finger. Moreover, the local wear on the main body can be reduced and the grip of the prosthetic hand can be improved by way of at least one reinforcing element on a sleeve portion and/or the palm, in particular on the thenar, the hypothenar and/or the distal region of the palm.

The main body of the cover for a prosthetic hand is advantageously hydrophobicized. In this way, the penetration of liquids or vapors into the prosthetic hand is effectively prevented and, moreover, the general hygiene of the prosthesis is improved. As an alternative or in addition, the main body can be provided with at least one other coating or surface treatment, for example such that the main body is oleophobic, fire-resistant and/or plasma coated.

One development provides that medial and/or lateral extensions which cover a joint in the proximal direction in the applied state are configured on at least one finger cap. Extensions of this type serve to protect particularly wear-susceptible regions of the main body and, moreover, also afford additional protection for the covered joint.

A fingernail component and/or a fingernail receiving region are/is preferably arranged or configured on the finger cap. As a result of the form of a fingernail component or a fingernail receiving region in an analogous manner with respect to the human fingernail, the natural appearance of the cover for a prosthetic hand is further improved. Moreover, there is the possibility for the user to attach individual fingernail components to the finger cap and/or to paint a fingernail component of this type. It is possible to carry out activities of daily life by way of a fingernail component or a fingernail, for example scraping off, scratching, lifting or the like of objects. Therefore, the fingernail components also afford further scope for individualizations of the cover for a prosthetic hand.

The finger cap advantageously has a homogeneous wall thickness. This embodiment affords the advantage of a consistent protective effect over the entire outer surface of the finger cap and, in addition, makes simpler manufacturing and mounting possible. As an alternative, an increased material thickness in comparison with a standard material thickness is provided on particularly loaded regions of the finger cap.

The finger cap preferably has a closed wall with a cavity for receiving a prosthetic finger. No locations, at which the protective effect is impaired, arise on the wall as a result. Moreover, manufacturing is simpler and, at the same time, the risk of molded part faults is lower. The form of a cavity makes it possible to put on the cover for a prosthetic hand without problems. Here, the finger caps provide an inherent stability which makes it possible for the cover for a prosthetic hand to be put on simply. The edge of the finger cap can be of contoured configuration, with the result that the cap ends further toward the distal end, for example, on the finger rear side than on the finger inner side.

The textile main body advantageously comprises a bi-elastic material or has bi-elastic portions. This material property makes it easier for the cover for a prosthetic hand to be put on and taken off, reduces the formation of folds, and facilitates resetting into a starting position.

The finger cap is preferably configured from a silicone, a PVC or another copolymer. The material selection of this type for the finger cap fosters a natural appearance. In addition, these materials afford a broad protective spectrum for the regions which are covered by the finger cap. The Shore hardness D 60 for PVC has proven highly suitable with regard to the durability and the flexibility. All the materials are preferably flexible.

One development provides that a recess in the main body is filled or covered by a silicone material, a PVC material or another copolymer. It is possible in this way for certain regions, in particular the thenar, hypothenar and/or the distal region of the palm, on the cover for a prosthetic hand to be provided with a more wear-resistant material and thus to prevent local wear on the main body.

In one refinement, the finger cap does not exclusively comprise a copolymer, but rather has a coating or a textile lining on the inner side of the finger cap, which coating or textile lining configures an air cushion between the prosthesis and the finger cap, as a result of which the cover for a prosthetic hand can be put on and taken off more easily.

In one variant, a slot or a fold with a closure device is arranged on the main body. The slot and the fold serve to introduce the prosthetic hand more easily into the cover for a prosthetic hand and to remove the cover for a prosthetic hand more easily. To this end, the closure device is opened, with the result that there is more space in the cover for a prosthetic hand. In the closed state of the closure device, the slot is closed or covered or the fold is folded in, with the result that the main body can bear tightly around the prosthetic hand or an adjoining component of the prosthesis, for example a forearm tube or a forearm shank. The closure device can be configured as a zipper, a hook and loop fastener or as a number of positively locking elements, for example as snap fasteners, hooks, buttons or the like. In this way, it is possible for the slot or the fold to be opened or folded open repeatedly and to be closed again and to be held closed.

In one refinement, the slot or the fold is arranged ulnarly and extends on the side of the ulna or the little finger side of the hand, in particular from the height of the ball of the hand in the proximal direction. The slot can run as far as the proximal end of the cover for a prosthetic hand, or else can configure a slot only in regions, which slot ends before the proximal end of the cover for a prosthetic hand.

In one refinement, at least one finger cap has a conductive component for operability of a touchscreen. By way of a conductive component of this type which can be inserted as a separate part or separate component in the finger cap or can be configured as a constituent part of the material of the finger cap, it is possible for capacitive operating surfaces or touchscreens to be operated simply. The conductive component is, for example, graphite, one or more conducting or conductive plastics, other conductive materials or a plasma coating.

The method for producing a cover as described above for a prosthetic hand provides that first of all a part glove with a main body is provided, and subsequently a separately produced finger cap with or made from a non-textile plastic is fastened to the main body as a replacement for a missing, distal, textile finger region on the part glove.

The finger cap is preferably produced in a vacuum casting, injection molding or additive manufacturing method. Using the vacuum casting and injection molding method, it is possible for finger caps to be manufactured with a consistent wall thickness and with very high precision. Moreover, the surface finish and the color can be configured easily. Additive manufacturing affords, in particular, advantages of a low lead time, inner and outer structures of virtually any desired complexity, and an inexpensive production of individualized finger caps which can be adapted, in particular, to the expectations of the user or to a certain prosthetic hand.

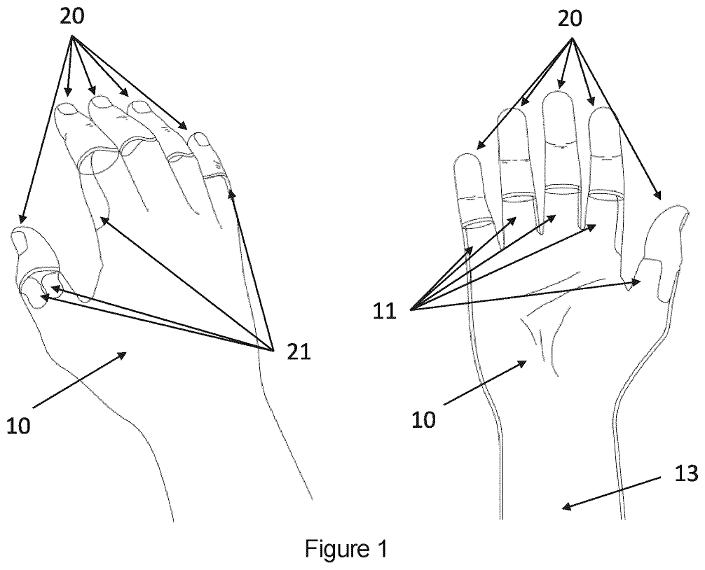

In the following text, one exemplary embodiment of the invention will be explained in greater detail on the basis of FIGS. 1 to 3. Identical designations denote identical components. In the figures:

FIG. 1—shows two views of the cover for a prosthetic hand with applied finger caps,

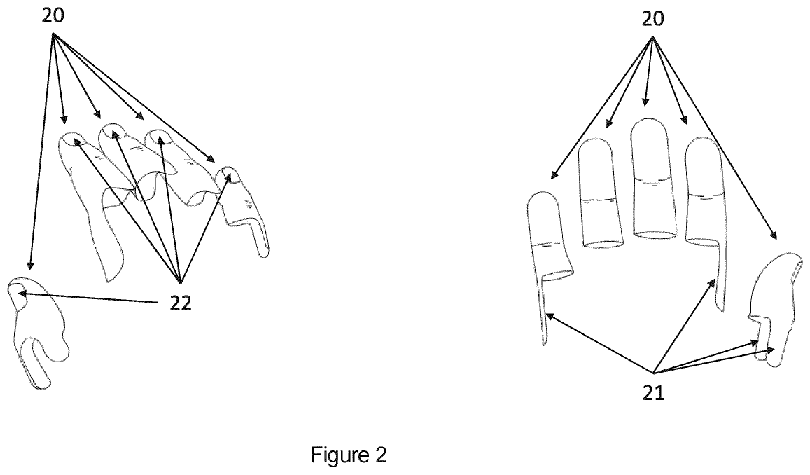

FIG. 2—shows an illustration of the finger caps alone,

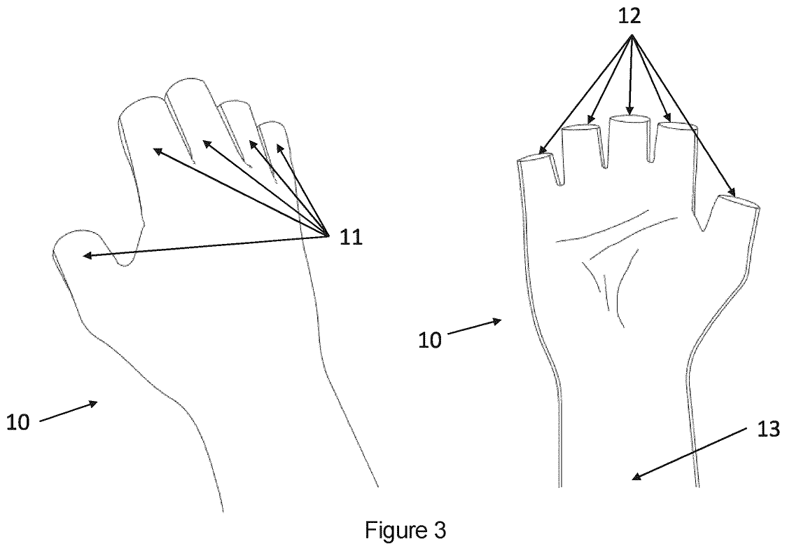

FIG. 3—shows an illustration of the textile main body,

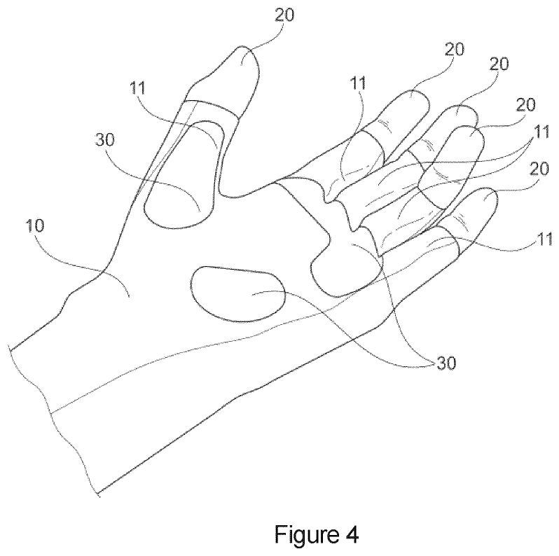

FIG. 4—shows a variant with reinforcing elements,

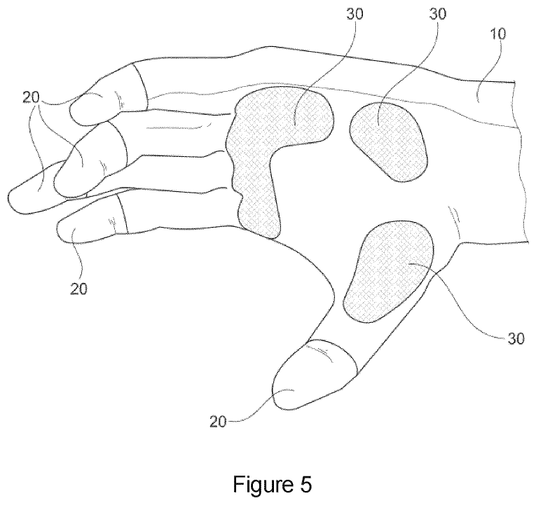

FIG. 5—shows a variant of FIG. 4, and

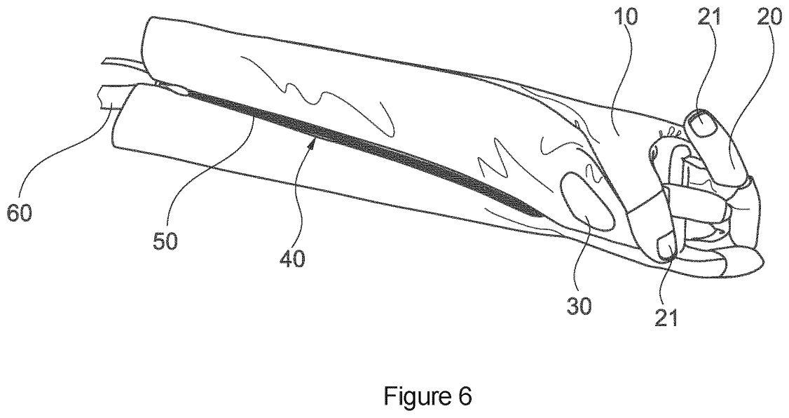

FIG. 6—shows an embodiment with a zipper.

FIG. 1 shows a cover for a prosthetic hand for a prosthetic right hand, which cover has a main body 10 and five finger caps 20. The textile main body 10 is configured as a part glove with passage openings at the distal end for guiding prosthetic fingers through. An opening 13 is configured at the proximal end of the main body 10, through which opening 13 the prosthetic hand can be introduced. A finger cap 20 made from a non-textile plastic, in particular a silicone material or a PVC material, is arranged in each case at the distal end of each finger, which finger cap 20 replaces the missing textile finger region and is connected to the main body 10. The textile main body 10 of the cover for a prosthetic hand extends in an applied state on each finger approximately as far as the position of the first finger joint of a prosthetic hand (not shown). To this end, sleeve portions 11 which cover at least the proximal phalanx of a prosthetic hand are configured on the main body 10 on each finger. The distal end of the sleeve portions 11 is of corresponding configuration with respect to the proximal end of the finger caps 20, with the result that the finger caps 20 can be sewn, adhesively bonded, cast and/or welded on the textile main body 10. As a result, in conjunction with the finger caps 20, the main body 10 configures a complete and closed cover for a prosthetic hand (not shown), which cover is adapted to the natural appearance of a hand. In the embodiment which is shown, the finger caps 20 configure substantially the distal phalanx in the case of the thumb and both the distal and the middle phalanx in the case of the remaining fingers. In the embodiment which is shown, the finger caps 20 of the thumb, the index finger and the little finger have medial and/or lateral extensions 21 which, in the applied state, cover a joint in the proximal direction and therefore protect against external mechanical effects on the joint. Both the main body 10 and the finger caps 20 have contours which correspond to the human hand and finger lines and skin folds.

The finger caps 20 can also be fastened releasably to the main body 10, for example via positively locking elements such as hook and loop fasteners, clamping strips, undercuts and the like. As an alternative, in one embodiment, the finger caps 20 are attached to the main body by way of an additive manufacturing method. To this end, the main body 10 is pulled on, for example, on a support in hand form, and the regions to be supplemented are integrally formed by way of 3D printing or similar methods and/or are thermoplastically adapted.

FIG. 2 shows the finger caps 20 of the cover for a prosthetic hand in an individual illustration. With their closed wall, the finger caps 20 each configure the distal and, if present, the middle phalanx. Here, the finger caps 20 have a homogeneous wall thickness, which can be achieved, in particular, by way of manufacturing with an accurate fit using the injection molding or vacuum casting method. The closed wall forms a cavity with an opening at the proximal end of the finger caps 20 for receiving a finger of a prosthetic hand (not shown). A fingernail component 22 is configured in each case on the finger caps 20, which fingernail component 22 is arranged in the vicinity of the distal end on the upper side of the fingers in an analogous manner with respect to the human hand, and therefore contributes to a natural appearance of the cover for a prosthetic hand. Instead of a complete fingernail, only a fingernail receiving region can also be configured on the finger cap 20, in which fingernail receiving region a separate fingernail is adhesively bonded, in order for it to be possible for a different look and different haptics to be used for a fingernail by way of a different material, for example acrylic. Medial and/or lateral extensions 21 are arranged on the thumb, the index finger and the little finger, which extensions 21 serve to protect the particularly wear-susceptible regions of the textile main body 10. As an alternative, medial and/or lateral extensions 21 can also be configured on each finger, in order to protect further joints against external influences, or extensions of this type are completely dispensed with.

The finger caps 20 can comprise a copolymer or, on their inner side, can have a coating or a textile cover which facilitates pulling on and taking off of the cover for a prosthetic hand.

FIG. 3 shows a main body 10 in an individual illustration. The main body 10 is configured as a part glove with passage openings 12 at the distal ends for receiving prosthetic fingers (not shown). The proximal end of the main body 10 has an opening 13 which serves to receive the prosthetic hand of the user. In the embodiment which is shown, the main body 10 covers the entire palm region and the back of the hand. As an alternative, the main body 10 can also be configured in such a way that, in the applied state, it only partially covers the palm region of a prosthetic hand (not shown). One advantage of the embodiment which is shown is that, in the applied state, the entire palm of the prosthetic hand is protected against contamination and other effects. To this end, the main body 10 is ideally hydrophobicized and/or coated in another way or adjusted with regard to its functional properties. A coating can improve the resistance capability against acids, oils, alkalis or other environmental influences, or can make a resistance capability of this type possible in the first case. The individual fingers are configured by way of the main body 10 in this embodiment only as far as a first joint as sleeve portions 11. Passage openings 12 are arranged in each case at the distal ends of the sleeve portions 11, through which passage openings 12 the individual fingers of a prosthetic hand (not shown) are guided when the cover for a prosthetic hand is put on, and to which the finger caps 20 are fastened.

FIG. 4 shows a perspective illustration of a cover for a prosthetic hand for a left prosthetic hand with a main body 10 and five finger caps 20 which are arranged at the distal ends of the sleeve portions 11. The finger caps 20 have a straight or a specifically curved proximal edge, and are fastened permanently to the textile main body 10, for example are welded on or adhesively bonded on. The proximal edge with curves can be seen in the illustrations of the finger caps 20 of FIGS. 1 and 2. The upper side and the lower side of the finger caps, and possibly also the lateral extensions on the index finger and the little finger, are extended, whereas the medial and bilateral portions are of curved or extended or punctiform configuration. As an alternative, the proximal edge is cut off in a straight line. The finger caps 20 are produced from a flexible, possibly elastic material, and supplement the sleeve portions 11 to form complete fingers, with the result that the cover for a prosthetic hand covers the prosthetic hand completely in the applied state. A cavity for receiving the distal ends of the prosthetic hand is configured in each case within the finger caps 20. Reinforcing elements 30 are fastened, for example molded, adhesively bonded, printed or cast, on the outer side of the textile main body 10 in the region of the palm, on the ball of the thumb, the ball of the hand and in the region of the base joints of the fingers. The reinforcing elements 30 can be produced separately and subsequently fastened to the textile main body 10, for example by way of activation of an adhesive using heat or pressure. The reinforcing elements 30 can comprise a textile or another material, and can also be connected permanently in a different way to the textile main body 10, for example via a hook and loop fastener or by being sewn on. The reinforcing elements can also cover or fill portions within the main body 10, for example in order to provide padding and wear protection on particularly loaded regions.

FIG. 5 shows a variant of FIG. 4, in the case of which a left-hand cover for a prosthetic hand with a plurality of reinforcing elements 30 on the palm is likewise depicted. The surface of the reinforcing elements 30 is profiled, in order for it to be possible for improved grip to be provided when gripping objects. The finger caps 20 on the distal end regions of the sleeve bodies 11 are produced from a base blank made from a closed, flat material. The respective base blanks have been shaped to form the finger caps 20, and have been fixed on the sleeve bodies 11 in order to complete the fingers.

FIG. 6 shows a further embodiment of the cover for a prosthetic hand for the left hand, in the case of which the textile main body 10 is extended in the proximal direction beyond the wrist and extends as far as into the region of the elbow. In the exemplary embodiment which is shown, finger caps 20 are fastened permanently to the main body 10 at all fingers. The finger caps 20 are configured from a flexible, in particular elastic material and, on the upper side, have fingernail components 21 which are fastened or configured in the fingernail region at the distal end of the finger caps 20.

In the exemplary embodiment which is shown, the textile main body 10 is provided with a slot 40 which is closed by a closure device 50 in the form of a zipper. The slot 40 extends from the wrist on the little finger side as far as the proximal end of the main body 10. In addition, a pull loop 60 is arranged on the main body 10, by way of which pull loop 60 it is possible for the cover for a prosthetic hand to be pulled over a prosthetic hand (not shown). As an alternative to the embodiment of the closure device 50 as a zipper, a plurality of snap fasteners, hooks, buttons or, for example, a hook and loop fastener can be arranged or configured on the main body 10, by way of which it is possible for the slot 40 to be opened or to be closed repeatedly. Instead of a slot 40, a fold can be incorporated in the forearm region or else in the region of the palm, for example ulnarly, which fold can be opened in order to pull on or pull off the cover for a prosthetic hand and can be folded in and closed after the introduction of the prosthetic hand into the cover for a prosthetic hand. By way of a combination of this type of a textile main body 10 with a possibly additional slot 40, the repeated pulling on and pulling off of the cover for a prosthetic hand is substantially facilitated, the finger caps 20 optionally with the fingernail components 21 ensuring an improved functionality and an improved durability.

### LIST OF DESIGNATIONS

10 Main body
11 Sleeve portion
12 Passage opening
13 Opening
20 Finger cap
21 Medial/lateral extension
22 Fingernail component
30 Reinforcing element

1. A cover for a prosthetic hand, comprising:
a textile main body which either covers or at least partially covers at least one palm region of a prosthetic hand in an applied state, wherein the textile main body is configured as a part glove with passage openings for prosthetic fingers; and
at least one distal finger cap comprising a non-textile plastic positioned at at least finger region associated with one of the passage openings.

2. The cover for a prosthetic hand as claimed in claim 1, wherein the at least one distal finger cap comprises a plurality of distal finger caps where each of the plurality of distal finger caps is positioned at a finger region associated with one of the passage openings.

3. The cover for a prosthetic hand as claimed in claim 1 wherein the at least one distal finger cap is sewn, adhesively bonded, cast, printed, and/or welded on the textile main body.

4. The cover for a prosthetic hand as claimed in claim 1 wherein the at least one distal finger cap is fastened removably and/or replaceably to the main body.

5. The cover for a prosthetic hand as claimed in claim 1 wherein the at least one distal finger cap is fastened to a textile sleeve portion of the main body, wherein the textile sleeve portion extends along a prosthetic finger in the applied state.

6. The cover for a prosthetic hand as claimed in claim 5, wherein the textile sleeve portion extends as far as a first or a second finger joint of the prosthetic finger.

7. The cover for a prosthetic hand as claimed in claim 1 further comprising at least one reinforcing element sewn, adhesively bonded, cast, printed, and/or welded on the textile main body.

8. The cover for a prosthetic hand as claimed in claim 1 wherein the textile one of the preceding claims, characterized in that the main body (10) is hydrophobicized.

9. The cover for a prosthetic hand as claimed in claim 1 further comprising medial and/or lateral extensions which cover a joint of a prosthetic finger in a proximal direction in an applied state are configured on the at least one distal finger cap.

10. The cover for a prosthetic hand as claimed in claim 1 further comprising a fingernail component and/or a fingernail receiving region are/is arranged or configured on the at least one distal finger cap.

11. The cover for a prosthetic hand as claimed in claim 1 wherein the at least one distal finger cap has a homogeneous wall thickness.

12. The cover for a prosthetic hand as claimed in claim 1 wherein the at least one distal finger cap comprises a closed wall with a cavity for receiving a prosthetic finger.

13. The cover for a prosthetic hand as claimed in claim 1 wherein the textile main body comprises a bi-elastic material or comprises bi-elastic portions.

14. The cover for a prosthetic hand as claimed in claim 1 wherein the at least one distal finger cap is configured from a silicone, a polyvinyl chloride (PVC), or a copolymer.

15. The cover for a prosthetic hand as claimed in claim 1 wherein the textile main body comprises a recess filled or covered by a silicone material or a polyvinyl chloride (PVC) material.

16. The cover for a prosthetic hand as claimed in claim 1 further comprising a coating or a textile lining arranged on an inner side of the at least one distal finger cap.

17. The cover for a prosthetic hand as claimed in claim 1 wherein the textile main body comprises a slot or a fold, and further comprising a closure device arranged at the slot or the fold of the textile main body.

18. The cover for a prosthetic hand as claimed in claim 17 wherein the slot or the fold is arranged to run from a height of a ball of a hand in a proximal direction.

19. The cover for a prosthetic hand as claimed in claim 1 further comprising a conductive component for operability of a touchscreen arranged on or together with the at least one distal finger cap.

20. A method for producing a cover for a prosthetic hand as claimed in claim 1, comprising one of the preceding claims with the steps:
providing a part glove with a main body, and
fastening a separately produced finger cap with a non-textile plastic to the main body as a replacement for a missing, distal, textile finger region.

21. The method as claimed in claim 20, wherein the finger cap is produced in a vacuum casting, injection molding, or additive manufacturing method.

22. The method as claimed in claim 20 the part glove is produced from a bi-elastic material.

23. The method as claimed in claim 20 wherein the finger cap is adhesively bonded, sewn, welded, printed, or cast onto the part glove.
